(19) United States
(12) Patent Application Publication
(10) Pub. No.: US 2014/0118686 A1
(43) Pub. Date: May 1, 2014

(54) IMAGING APPARATUS AND FOCUSING METHOD FOR IMAGING APPARATUS

(71) Applicant: CANON KABUSHIKI KAISHA, Tokyo (JP)

(72) Inventors: Toshiya Fujimori, Kawasaki-shi (JP); Nobuyoshi Kishida, Nishitokyo-shi (JP)

(73) Assignee: CANON KABUSHIKI KAISHA, Tokyo (JP)

(21) Appl. No.: 14/049,490

(22) Filed: Oct. 9, 2013

(30) Foreign Application Priority Data
Oct. 26, 2012 (JP) 2012-236358

(51) Int. Cl. A61B 3/14

(52) U.S. Cl. CPC A61B 3/14
USPC 351/206

(57) ABSTRACT

Provided is an ophthalmologic apparatus having an automatic focusing function and capable of imaging a subject in focus, including: a light intensity control unit for controlling a light intensity of light guided to an object to be inspected; an imaging unit for imaging the object to be inspected which is illuminated by the light; a focus state detection unit for detecting a focus state of the imaging unit with respect to the object to be inspected based on an output from the imaging unit; a focus lens drive unit for driving a focus lens based on the focus state detected by the focus state detection unit; and a drive control unit for controlling the focus lens drive unit to operate in accordance with timing when the light intensity of the light is changed by the light intensity control unit.

### BACKGROUND OF THE INVENTION

1. Field of the Invention

The present invention relates to an imaging apparatus, for example, an ophthalmologic imaging apparatus having an automatic focusing function, such as a fundus camera used by an ophthalmology hospital, and a mass screening that observes or photographs a fundus of an eye to be inspected, and to a focusing method for an imaging apparatus, such as an ophthalmologic imaging apparatus.

2. Description of the Related Art

Heretofore, an automatic focusing apparatus has widely adopted a contrast detection system configured to detect contrast of a video signal of a subject image to perform a focus operation. The contrast detection system utilizes such characteristics that the contrast of the video signal becomes higher as the lens is being brought into focus. That is, the system is a control system in which an optimum lens position is searched for by detecting a focus lens position corresponding to a peak of this contrast, value while moving the focus lens. As an element that converts an optical signal of the subject image into an electrical video signal, a solid-state image pickup element, such as a CCD and a CMOS, is used, and a contrast value can be obtained by detecting a high frequency component from this video signal.

Japanese Patent Application Laid-Open No. 2006-280477, as illustrated in FIGS. 1A to 1C, proposes a fundus camera that determines the contrast of the outline of a focus index image 190a′ or the outline of a focus index optical image and determines a focus state thereof by the focus lens, to thereby perform automatic focusing.

Incidentally, almost all cameras used as imaging cameras in the ophthalmologic imaging apparatus are commonly used digital cameras.

Further, Japanese Patent Application Laid-Open No. 2008-276131 proposes a method for accurately obtaining a focusing point, in an automatic focusing operation of a contrast system in a manner that a camera control portion transmits a timing signal to a lens control portion and the lens control portion obtains a focus lens position synchronously with the timing signal.

The automatic focusing operation of the contrast system synchronizes the camera control portion and the lens control portion by transmitting the timing signal to the lens control portion from the camera control portion. However, the system becomes complicated because an automatic focusing evaluation value needs to be calculated by exposing at timing related to the timing signal, the focus lens position needs to be detected in accordance with the acquisition of the timing signal, and the evaluation value and the focus lens position need to be associated. In addition, when the timing signal is not received, the evaluation value and the focus lens position cannot be accurately associated, and hence a subject, which is not brought into focus, may be photographed.

Furthers almost ail cameras used as imaging cameras in the ophthalmologic imaging apparatus are commonly used digital cameras. In the digital camera and the ophthalmologic apparatus, it is difficult to accurately associate a contrast evaluation value with the focus lens position unless there is a synchronizing signal from the digital camera. Even if the synchronizing signal is received from the digital camera, the structure that obtains the synchronizing signal from the digital camera becomes complicated in the ophthalmologic apparatus, and therefore, the apparatus becomes expensive.

### SUMMARY OF THE INVENTION

The present invention has been made in view of the above-mentioned problems, and is to provide an imaging apparatus and a focusing method for an imaging apparatus, which are capable of associating a focus evaluation value and a focus lens position with, each other by a simple structure and thus obtaining a subject image in focus.

In order to solve the above-mentioned problems, an ophthalmologic imaging apparatus having an automatic focusing function according to one embodiment of the present invention includes: a light intensity control unit for controlling a light intensity of light guided to an object to be inspected; an imaging unit for imaging the object to be inspected which is illuminated by the light; a focus state detection unit for detecting a focus state of the imaging unit with respect to the object to be inspected based on an output from the imaging unit; a focus lens drive unit for driving a focus lens based on the focus state detected by the focus state detection unit; and a drive control unit, for controlling the focus tens drive unit to operate in accordance with timing when the light intensity of the light is changed by the light intensity control unit.

According to the present invention, the contrast evaluation value and the focus lens position can be associated with each other and thus a subject image in focus can be photographed.

Further features of the present invention will become apparent from the following description of exemplary embodiments with reference to the attached drawings.

### BRIEF DESCRIPTION OF THE DRAWINGS

FIGS. 1A, 1B and 1C are diagrams representing the content disclosed in Japanese Parent Application Laid-open No. 2006-280477.

FIG. 2 is a configuration diagram of an ophthalmologic imaging apparatus according to a first embodiment of the present invention.

FIG. 3 is a configuration diagram of a focus detection portion in the first embodiment of the present invention.

FIG. 4 is a view enlargedly showing a fundus image projected to a monitor.

FIG. 5 is a graph showing focus position detection by the focus detection portion in the first embodiment of the present invention.

FIGS. 6A, 6B, 6C, 6D, 6E and 6F are diagrams showing principles of contrast detection.

FIG. 7 is a characteristic graph showing focus position detection by the focus detection portion when an observation light intensity is dimmed in the first embodiment of the present invention.

FIG. 8 is a flowchart of a characteristic control method in the first embodiment of the present invention.

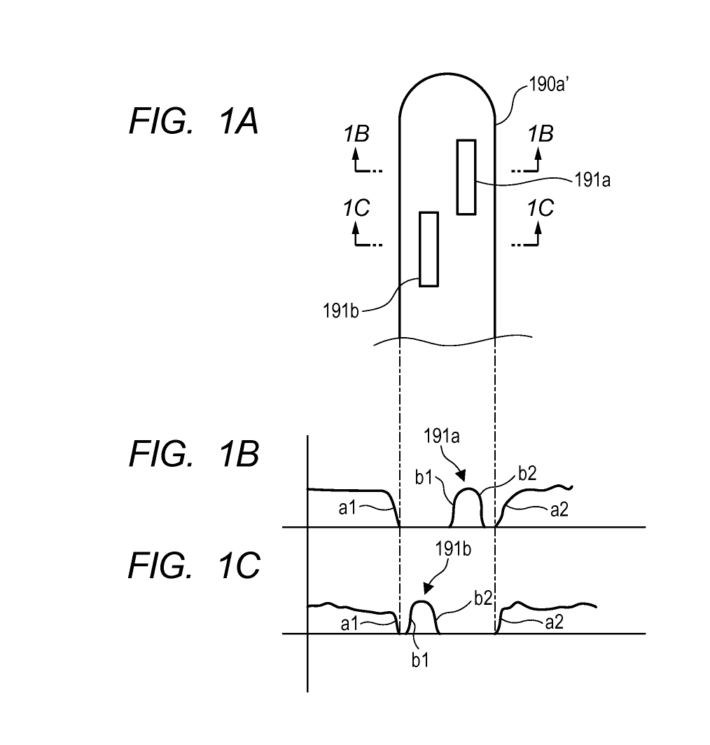

### DESCRIPTION OF THE EMBODIMENTS

The present invention is described in detail with reference to the illustrated embodiments.

### First Embodiment

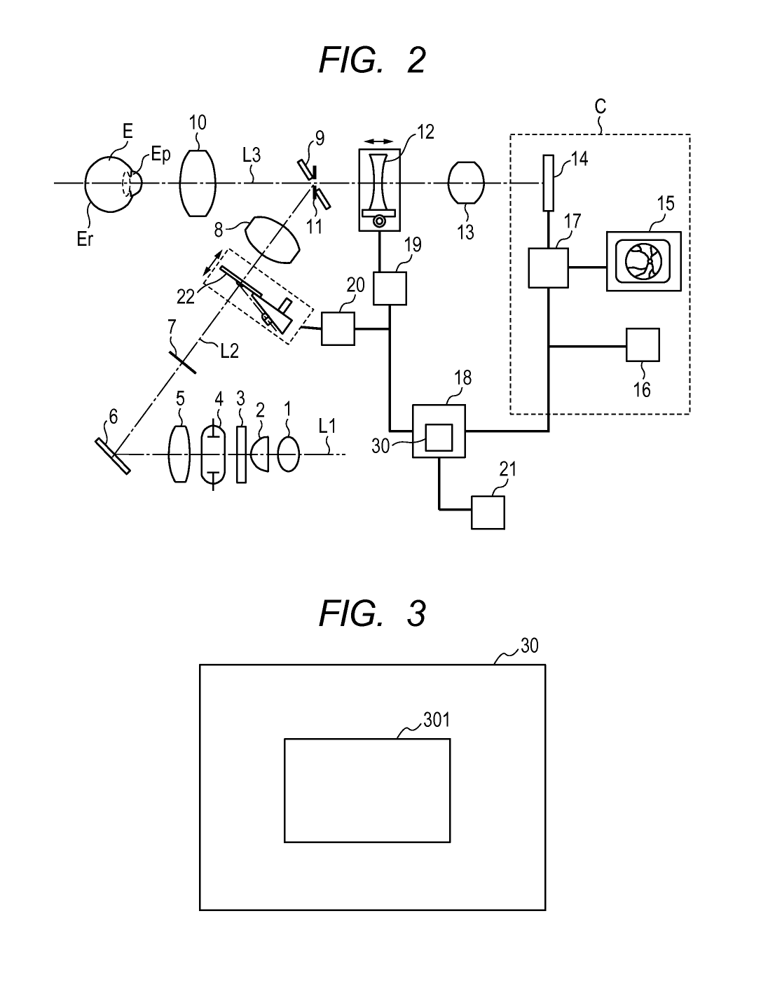

FIG. 2 illustrates a first configuration example of a fundus camera which is an ophthalmologic imaging apparatus embodying the present invention.

There are arranged on an optical axis L1 an observation light source 1 for emitting stationary light such as a halogen lamp, a condenser lens 2, a filter 3 transmitting infrared light and blocking visible light, an imaging light source 4 such as an electronic flash, a lens 5, and a mirror 6. Further, there are sequentially arranged on an optical axis L2 in the direction of reflection of the mirror 6 a ring diaphragm 7 having a ring-shaped, opening, a relay lens 8, and a perforated mirror 9 having a central portion opening. These elements serve as a unit for observing a fundus.

Further, an objective lens 10 placed to face an eye E to be inspected is arranged on an optical axis L3 in the direction of reflection of the perforated mirror 9. A hole of the perforated mirror 9 is sequentially arranged with an imaging diaphragm 11, a focus lens 12 for adjusting focus by moving the position on the optical axis L3, and an imaging lens 13. Ahead of the imaging lens 13, there is sequentially arranged an image pickup element 14 not only for observing moving images but also for imaging still images within an imaging camera C. An output of the image pickup element 14 is connected to an image processing portion 17, and an output of the image processing portion 17 is connected to a system control portion 18. The image processing portion 17 projects an observation image photographed by the image pickup element 14 to a monitor 15. These elements serve as a unit for imaging the fundus.

Meanwhile, a focus index projection portion 22 is arranged between the ring diaphragm 7 and the relay lens 8 on the optical axis L2. Note that, the focus index projection portion 22 and the focus lens 12 are respectively moved simultaneously in the direction of the optical axis L2 and the optical axis L3 by a focus lens drive portion 19 and a focus index drive portion 20 based on the control from the system control portion 18. This system control portion 18 controls the focus lens drive portion 19 and the focus index drive portion 20 in accordance with the operation input of an operation input portion 21 in a manual focusing mode. At this time, the focus index projection portion 22 and the image pickup element 14 have optically a conjugate relation. In addition, the system control portion 18 controls the focus lens drive portion 19 and the focus index drive portion 20 based on the detection result of a focus detection portion 30 within the system control portion 18 in an automatic focusing mode.

Further, the system control portion 18 performs a control of the light intensity adjustment, and turning-ON/OFF of the observation light source 1 and also a control, of the light intensity adjustment and turning-ON/OFF of the imaging light source 4. Note that, these light sources can share their functions, and the system control portion 18 for controlling the light intensity adjustment and turning-ON/OFF of the light sources includes the area that functions as a light intensity control unit for controlling the light intensity of light which is guided 10 the fundus of an eye to be inspected in the present invention.

Next, operations in this embodiment are described.

The system control portion 18 turns on the observation light source 1. A light beam, emitted from the observation light source 1 is collected by the condenser lens 2. The visible light thereof is cut off by the filter 3, and only the infrared light thereof is transmitted. Then, the light beam transmits through the imaging light source 4 such as the electronic flash so as to be transformed into a ring light beam via the lens 5, the mirror 6, and the ring diaphragm 7. After this, the light beam is deflected in the direction of the optical axis L3 by the relay lens 8 and the perforated mirror 9, and illuminates a fundus Er of the eye E to be inspected via the objective lens 10. The light beam that reaches the fundus Er is reflected and scattered, and is emitted from the eye E to be inspected and imaged on the image pickup element 14 after passing through the objective lens 10, the imaging diaphragm 11, the focus lens 12, and the imaging lens 13. Then, the system control portion 18 projects a fundus image photographed by the image pickup element 14 to the monitor 15.

An operator performs the fine adjustment of the positioning between the eye E to be inspected and an optical unit formed of the above-mentioned optical elements while observing the fundus image projected to the monitor 15, and then, after performing focus adjustment, performs imaging by pushing an imaging switch (not shown). The apparatus described in this embodiment has an automatic focusing function of automatically executing this focus adjustment.

Next, the focus detection portion 30 which is a constituent element of the first embodiment is described with reference to FIG. 3. The focus detection portion 30 contains a contrast detection portion 301 which is used for focusing. The contrast detection portion 301 is connected to the image pickup element 14 for observation image.

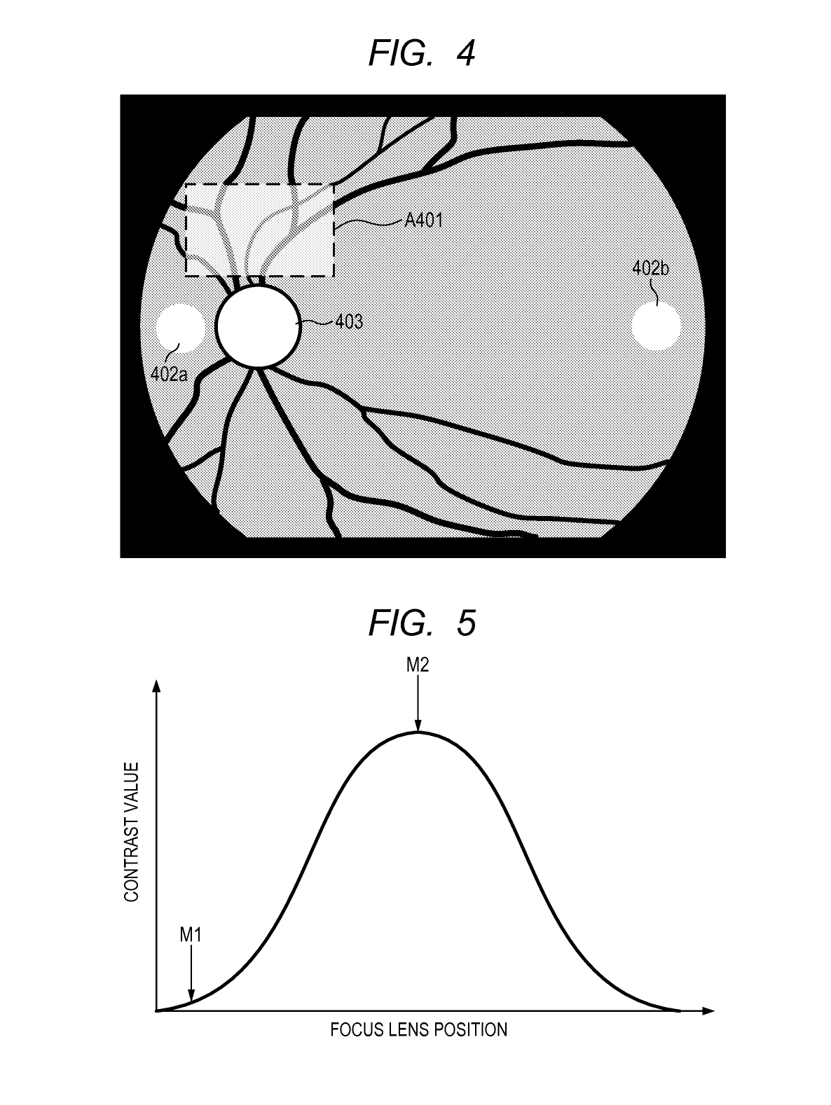

Next, the subject detected by the contrast defection portion 301 is described with reference to FIG. 4. FIG. 4 is a view enlargedly showing the fundus image projected to the monitor 15. An area A401 in the figure is a focus detection position and a range of the contrast detection portion. Note that, images 402a and 402b in the figure represent alignment index images for performing the positioning between the fundus camera and the eye to be inspected. Further, an image 403 in the figure represents a papillary portion of the fundus. These alignment index images 402a and 402b and the papillary portion are not characteristic points in this embodiment, and therefore, the detailed description thereof is omitted.

Now, the contrast detection portion 301 is described in detail with reference to FIG. 5.

First, a focus detection range implemented in the contrast detection portion 301 is a medium and large artery on the retina in the area A401 of FIG. 4. The graph shown in FIG. 5 represents a transition in a contrast value for the position of the focus lens 12 which is moved by the focus lens drive portion 19.

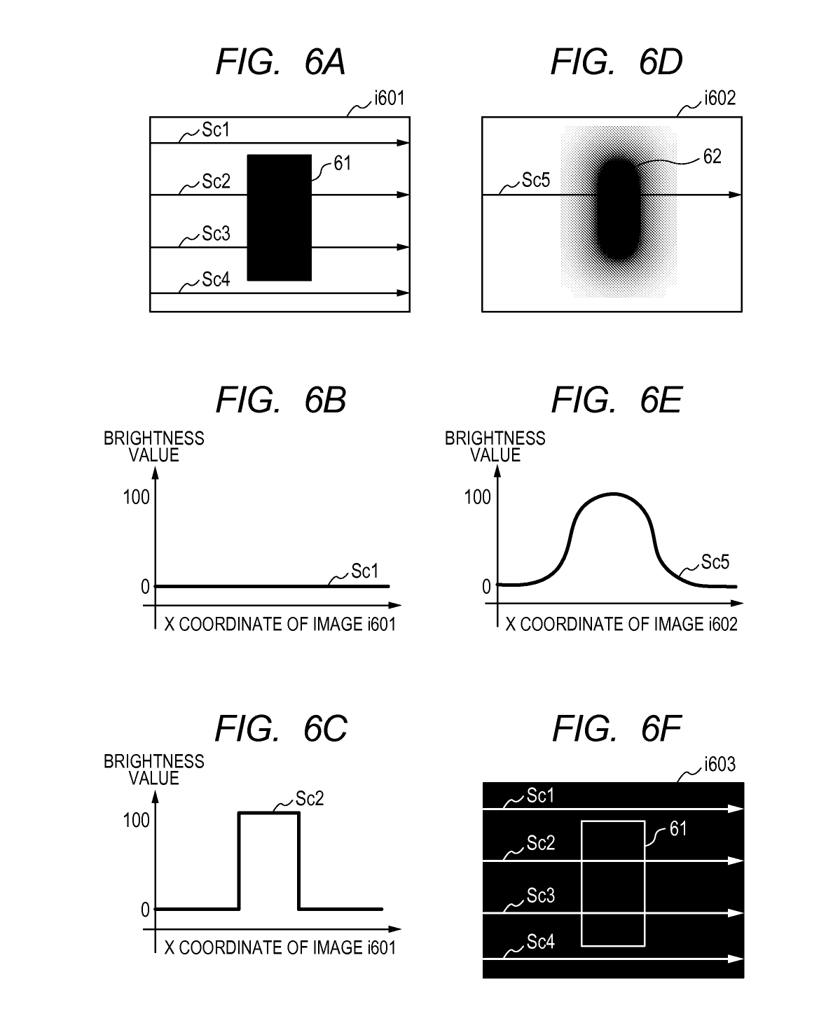

Next, the calculation method of the contrast value is described with reference to FIGS. 6A to 6F. Here, the contrast means a brightness difference between adjacent pixels, and the contrast value means a value of the largest brightness difference in brightness data of a scan line. Further, the arrow marks of scan lines Sc1 to Sc4 represent directions thereof, and scan the lines in the horizontal direction, the lines corresponding to the number of images in the vertical direction from the upper part to the lower part in accordance with an image size of an image i601 as exemplified in FIG. 6A. The contrast value of the entire image i601 is calculated as a sum of the contrast values obtained for individual lines by scanning the lines corresponding to the number of images in the vertical direction from the upper part to the lower part.

For easy understanding, for example, brightness of a part of an image 61 in the image i601 is set to 100, and brightness of a part other than the image is set to 0. First, in the scan line Sc1, the image 61 is not included in the scan line, and hence all brightness are identical to each other, and as a result, the contrast value in the scan line Sc1 is calculated as 0. FIG. 6B represents a brightness change seen from the scan line Sc1, and a state where the contrast value in the scan line Sc1 is calculated as 0 is recognized. Next, in the scan line Sc1, the image 61 is included in the scan line, and hence a difference between brightness of the part other than the image and brightness of the left side face of the image 61 is calculated as the contrast value in the scan line Sc2. Here, the difference between both brightness is 100, and hence the contrast value is 100. FIG. 6C represents the brightness change seen from the scan line Sc2.

Next, the contrast value of the entire image i601 is calculated as a sum of the contrast values obtained for individual lines by scanning the lines corresponding to the number of images in the vertical direction from the upper part to the lower part. For example, assuming that a longitudinal length of one image 61 corresponds to ten scan lines, the length is expressed by 100×10=1,000.

In this manner, the contrast detection portion 301 obtains the contrast, value of the entire image.

Next, relationships between, the contrast value and focusing are described.

An image 62 in an image i602 as exemplified in FIG. 6D is an image that defocuses the image 61 in the image i601. When looking at FIG. 6E that represents the brightness change seen, from the scan line Sc5 in the image i602, waveforms thereof ere seen to be deformed as compared to the brightness change of the scan line Sc2. Following the calculation method of the contrast value already described, because the contrast value is the largest value of the brightness difference between adjacent pixels, the scan line Sc5 has a smaller contrast value calculated than the scan line Sc2.

In this manner, when the image exists in the best focus state, the contrast value is large, and as the image is gradually defocused, the contrast value becomes smaller.

In FIG. 5, the brightness difference between the part other than the medium and large artery and both end parts of the medium and large artery on the retina is calculated as the contrast value. In a focus position M2 which exists in the best focus state, the brightness difference between the part other than the medium and large artery and both end parts of the medium and large artery is large, and hence the contrast value becomes the maximum. In a position M1 which is greatly defocused, the brightness difference between the part other than the medium and large artery and both end parts of the medium and large artery is small, and hence the contrast value becomes small.

Next, relationships between driving of the conventional focus lens and the contrast value are described.

In the automatic focusing of the conventional contrast system, as described in Japanese Patent Application Laid-open No. 2008-276131, the focus lens is driven synchronously with a synchronizing signal which is exposure start timing of the optical signal in an imaging unit so that the contrast evaluation value and the focus lens position are favorably grasped. However, when the synchronizing signal is not received, the contrast evaluation value and the focus lens position cannot be favorably grasped. Hence, the contrast evaluation value and the focus lens position cannot be associated, and hence the subject not brought into focus may be photographed.

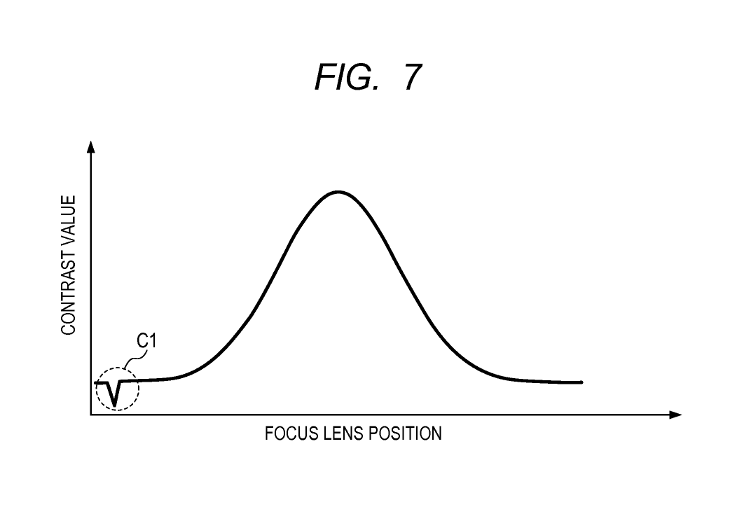

Now, a characteristic control in this embodiment is described with reference to FIG. 7.

FIG. 7 represents a transition in the contrast value for the position of the focus lens 12 which is moved by the focus lens drive portion 19. A characteristic point C1 in FIG. 7 shows that the observation light source 1 is dimmed and the contrast value calculated by the contrast detection portion 301 is allowed to calculate a characteristic point.

For easy understanding, the contrast evaluation value when the observation light source 1 is turned off and the image 61 in the image i601 is taken as a focus object is described with reference to the image i601 of FIG. 6A and an image i603 of FIG. 6F.

The calculation method of the contrast evaluation value is as described earlier. Assuming that brightness of the part of the image 61 in the image i601 is set to 100, brightness of the part other than the image is set to 0, and the longitudinal length of one image 61 corresponds to ten scan lines, the contrast evaluation value is expressed by 100×10=1,000. However, as shown in the image i603, when the observation light source 1 is turned off, brightness of the image 61 and brightness of the part other than the image 61 become equal, and the contrast evaluation value becomes 0. Note that, if the dimming time is too long, areas are formed where the contrast value sequentially changes. Thus, a time in which the light intensity of illumination light is changed such as the dimming is preferably set to a time shorter than a frame rate when the contrast evaluation value is detected.

Execution of the above-mentioned operations can intentionally produce the characteristic point in the contrast evaluation value. Further, in the embodiment, though a case where the observation light source 1 is turned off is described, the characteristic point can be produced in the contrast evaluation value even when the observation light source is flashed or increased, in light intensity. In addition, it is self-evident that the characteristic point can be calculated by using the brightness evaluation value ins Lead of the contrast value.

Next, start timing of driving of the focus lens is described.

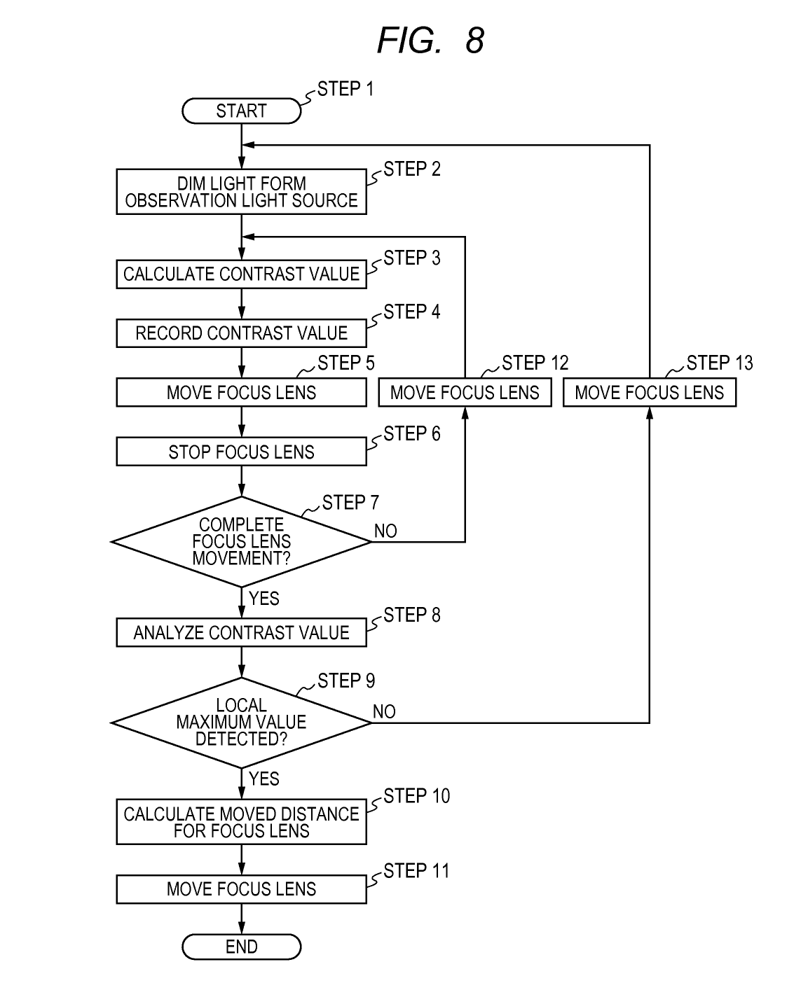

Start of driving of the focus lens may be performed at optional timing after the observation light source 1 is dimmed and the characteristic point is produced in the contrast evaluation value calculated by the contrast detection portion 301. Further, after the light source is dimmed and a predetermined period elapses, driving of the focus lens may be performed. FIG. 8 illustrates a flowchart where, for example, the observation light source 1 is dimmed and driving of the focus lens is started after 100 ms.

First, in Step 1, the focus detection for the medium and large arteries of the fundus is started. In Step 2, the observation light source 1 is dimmed by an observation light intensity control unit. Step 3 is executed by the contrast detection portion 301 to calculate the contrast, and Step 4 is executed by the contrast detection portion 301 to record the value calculated in Step 3. Step 5 is executed by a focus lens drive unit to drive the focus lens by a predetermined amount. As described above, if Step 1 to Step 5 are executed in 100 ms, the characteristic point is produced in the contrast value, and accurate start timing of the focus drive can be known. In Step 6, the focus lens is temporarily stopped by the focus lens drive portion 19 which is the focus lens drive unit. When driving of the focus lens is not terminated in Step 7, the focus lens is driven again by the predetermined amount. Here, the focus lens drive amount means a predetermined amount to be driven in one direction.

When, driving of the focus lens is terminated in Step 7, the procedure advances to Step 8.

In Step 8, analysis is executed starting from the characteristic point recorded with the contrast value by the contrast detection portion 301. First, the characteristic point calculated when the observation, light source 1 is dimmed in Step 2 is detected, and then, the presence or absence of the local maximum value of the contrast value is determined.

Step 9 is executed by the contrast detection portion 301, and a condition branch is performed. Here, when the local maximum value is detected, the procedure advances to Step 10. Step 10 is executed by the contrast detection portion 301 to calculate the moved distance of the focus lens. Here, the moved distance of the focus lens in Step 10 means a drive amount of the focus lens up to the detection position of the local maximum value. Next, in Step 11, driving of the focus lens is performed in accordance with the moved distance of the focus lens calculated in Step 10, and the position, of the focus lens 12 is moved to the position of the local maximum, value of the contrast value.

When the local maximum value is not detected in Step 9, the procedure advances to Step 13. Here, the moved distance of the focus lens in Step 13 means a drive amount of the focus lens up to a focus detection start position on the medium and large arteries of the fundus in Step 1.

Such operations are particularly effective in the fundus camera where the focus lens position and the contrast value cannot be connected by the reception of the exposure start synchronizing signal of the optical signal in the imaging unit when the automatic focusing is performed by the contrast system. Even when the fundus camera is unable to receive the synchronizing signal from the imaging unit, the characteristic point can foe produced in the contrast value by changing the intensity of the observation light which illuminates the eye to be inspected. Hence, if driving of the focus lens is performed when a predetermined period elapses from timing of changing the observation light intensity, start timing of driving of the focus lens can be known, and the position of the focus lens and the contrast value can be connected. Further, it is self-evident that the characteristic point can be calculated by using the brightness evaluation value instead of the contrast value.

Note that, in the above-mentioned configuration, the image pickup element 14 corresponds to the imaging unit for imaging the fundus illuminated by light and the contrast detection portion 301 corresponds to a focus state detection unit for detecting a focus state based on the output of the imaging unit. Further, the system control portion 18 also includes an area which functions as a drive control unit in the present invention which controls the focus lens drive unit to operate in accordance with timing when the light intensity is changed by the light intensity control unit.

Further, if the contrast value can be immediately obtained even when the synchronizing signal cannot be received, the contrast evaluation value can he accurately associated with the focus lens position. Note that, it is necessary to perform communications between the system control portion 18 and the focus detection portion 30 in order to obtain the contrast value. For this reason, a time lag is generated. Consequently, it is difficult to obtain the contrast value immediately in this case.

As described above, in the ophthalmologic imaging apparatus having the automatic focusing function according to the present invention, the observation light intensity is dimmed by the observation light intensity control unit before the focus lens is driven by the focus lens drive unit. With this, the characteristic point can be produced in the contrast evaluation value detected by the focus state detection unit, and hence start timing of driving of the focus lens can be accurately known. Consequently, even if the ophthalmologic imaging apparatus is provided with an imaging camera separately, the contrast evaluation value and the focus lens position can be accurately associated, and the subject brought into focus can be photographed. Further, because there is no configuration required for detecting a timing signal from the imaging camera provided separately, the structure of the apparatus can be simplified, and a small-sized inexpensive apparatus can be provided.

### Other Embodiment

Further, the present invention may also be realized by executing the following process. Specifically, software (program) for realizing the function of the embodiment described above is supplied to a system or an apparatus via a network or an arbitrary type of storage medium, and a computer (CPU or MPU) of the system, or the apparatus reads and executes the program.

While the present, invention is not limited to the above-mentioned embodiments, various modifications and changes may be made within the scope without departing from the spirit of the present invention. For example, as the above-mentioned embodiments describes a case where an object to be measured is an eye, the present invention may be applied also to an object to be measured such as the skin and the organ other than the eye. In this case, the present invention includes an embodiment as medical equipment other than the ophthalmologic apparatus, for example, an endoscope. Consequently, the present invention is desirably grasped as an inspection apparatus as exemplified by the ophthalmologic apparatus, and an eye to be inspected and a fundus thereof are desirably grasped as an embodiment of the object to be inspected.

While the present invention has been described with reference to exemplary embodiments, it is to be understood that the invention is not limited to the disclosed exemplary embodiments. The scope of the following claims is to be accorded the broadest interpretation so as to encompass ail such modifications and equivalent structures and functions.

This application claims the benefit of Japanese Patent Application No. 2012-236358, filed Oct. 26 2012, which is hereby incorporated by reference herein in its entirety.

What is claimed is:

1. An ophthalmologic imaging apparatus having an automatic focusing function, comprising:
a light intensity control unit for controlling a light intensity of light guided to an object to be inspected;
a imaging unit for imaging the object to be inspected which is illuminated by the light;
a focus state detection unit for detecting a focus state of the imaging unit with respect to the object to be inspected based on an output from the imaging unit;
a focus lens drive unit for driving a focus lens based on the focus state detected by the focus state detection unit; and
a drive control unit for controlling the focus lens drive unit to operate in accordance with timing when the light intensity of the light is changed by the light intensity control unit.

2. An ophthalmologic imaging apparatus according to claim 1, wherein the drive control unit controls the focus lens drive unit to drive the focus lens after a predetermined period elapses after the light intensity is changed by the light intensity control unit.

3. An ophthalmologic imaging apparatus according to claim 1, wherein the imaging unit comprises a unit for observing the object to be inspected and a unit for imaging the object to be inspected.

4. An ophthalmologic imaging apparatus according to claim 1, wherein the light intensity control unit changes the light intensity by dimming a light source for emitting the light.

5. An ophthalmologic imaging apparatus according to claim 1, wherein:
the focus state detection unit detects the focus state by a contrast evaluation value based on contrast in an image of the object to be inspected obtained by the imaging unit; and
the light intensity control unit changes the light intensity in a period shorter than a frame rate which detects the contrast evaluation value,

6. A focusing method for an ophthalmologic apparatus,
the ophthalmologic apparatus being configured to:
guide light, to an object to be inspected;
photograph the object to be inspected illuminated by the light with a imaging unit;
detect a focus state of the imaging unit with respect to the object to be inspected based on an output from the imaging unit; and
drive a focus lens based on the detected focus state,
the focusing method comprising changing a light intensity of the light when the light is guided to the object to be inspected, and starting to drive the focus lens in accordance with timing of changing the light intensity.

7. A program for controlling a computer to execute the steps of the focusing method according to claim 6.
